(19) United States
(12) Patent Application Publication
(10) Pub. No.: US 2024/0124834 A1
(43) Pub. Date: Apr. 18, 2024

(54) CELL SHEET SUPPORT, CELL SHEET LAMINATE AND METHOD FOR PRODUCING SAME

(71) Applicant: FUKUOKA UNIVERSITY, Fukuoka (JP)

(72) Inventors: Mai HAZEKAWA, Fukuoka-shi, Fukuoka (JP); Shigeru YAO, Fukuoka-shi, Fukuoka (JP)

(73) Assignee: FUKUOKA UNIVERSITY, Fukuoka (JP)

(21) Appl. No.: 18/277,278

(22) PCT Filed: Feb. 15, 2022
(86) PCT No.: PCT/JP2022/005967

§ 371 Date: Aug. 15, 2023

(30) Foreign Application Priority Data
Feb. 17, 2021 (JP) 2021-023164

(51) Int. Cl. C12N 5/00

(52) U.S. Cl. CPC C12N 5/0068; C12N 2533/30; C12N 2539/10

(57) ABSTRACT

Provided is a cell sheet support that satisfactory adheres to cultured cell and has biodegradability. The cell sheet support contains a first polymer containing a structural unit derived from p-dioxanone. The cell sheet support further contains a second polymer containing at least one selected from the group consisting of polylactic acid, polyglycolic acid, polycaproic acid, and copolymers thereof. The content ratio of the first polymer to the total amount of the first polymer and the second polymer is not less than 50% by mass. The cell sheet support has a sheet shape having an average thickness of 10 μm to 150 μm.

### TECHNICAL FIELD

The present invention relates to a cell sheet support, a cell sheet laminate, and methods of producing them.

### BACKGROUND ART

Transplantation methods using cell sheets have been developed to regenerate damaged biological tissues. Many of the cell sheets used for the transplantation are prepared especially from anchorage-dependent cells, among animal cells including human cells. For the production of a cell sheet, animal cells need to be allowed to adhere to a surface of a substrate, and to be cultured into a sheet shape. The cells then need to be detached while the shape is maintained. For example, WO 02/08387 proposes a method in which cells are cultured on a cell culture support prepared by coating a substrate surface with a polymer having an upper or lower critical solution temperature of 0° C. to 80° C. to water, to obtain a cell sheet, which cell sheet is then allowed to adhere to a polymer membrane, followed by changing the culture liquid temperature to not less than the upper critical solution temperature or not more than the lower critical solution temperature to detach the cell sheet together with the polymer membrane. Further, a cultured cell sheet supported by a biodegradable polyester porous membrane has been proposed in Membrane, 40(3), 124-129 (2015).

### SUMMARY OF THE INVENTION

### Problems to be Solved by the Invention

In the polymer membrane described in WO 02/08387, its insufficient adhesion to cells may lead to insufficient recovery of the cultured cells. Moreover, the polymer membrane needs to be detached from the cell sheet after the cell sheet is transferred to the desired site. An object of the present invention is to provide a cell sheet support showing good adhesion to cultured cells and having biodegradability.

### Means for Solving the Problems

The following are specific means to solve the above problem, and the present invention includes the following aspects. A first embodiment is a cell sheet support comprising a first polymer containing a structural unit derived from p-dioxanone. The cell sheet support may comprise a second polymer containing at least one selected from the group consisting of polylactic acid, polyglycolic acid, polycaproic acid, and copolymers thereof. The content ratio of the first polymer to the total amount of the first polymer and the second polymer may be not less than 50% by mass. The cell sheet support may have a sheet shape having an average thickness of not less than 10 μm and not more than 150 μm. The cell sheet support may be used for transferring a cell sheet, or may be used for layering a cell sheet.

A second embodiment is a cell sheet laminate comprising: the cell sheet support; and a cell sheet placed on the cell sheet support.

A third aspect is a method of producing a cell sheet laminate, the method comprising: culturing cells on a temperature-responsive polymer layer, to provide a cell sheet; layering the cell sheet support on the cell sheet; and detaching the cell sheet from the temperature-responsive polymer layer by a temperature change, to obtain a cell sheet support having the cell sheet adhering thereto.

A fourth embodiment is a method of transferring a cell sheet, the method comprising: culturing cells on a temperature-responsive polymer layer, to provide a cell sheet; layering the cell sheet support on the cell sheet; detaching the cell sheet from the temperature-responsive polymer layer by a temperature change, to obtain a cell sheet support having the cell sheet adhering thereto, and bringing the cell sheet on the cell sheet support into contact with a target site.

A fifth embodiment is a method of producing a cell sheet laminate, the method comprising forming a cell sheet by culturing cells on the cell sheet support having a surface on which a hydrophilic coating layer containing a hydrophilic polymer is formed.

A sixth embodiment is a method of transferring a cell sheet, the method comprising: forming a cell sheet by culturing cells on the cell sheet support having a surface on which a hydrophilic coating layer containing a hydrophilic polymer is formed; and bringing the cell sheet on the cell sheet support into contact with a target site.

### Advantageous Effects of the Invention

According to the present invention, a cell sheet support showing good adhesion to cultured cells and having biodegradability may be provided.

### BRIEF DESCRIPTION OF THE DRAWINGS

FIG. 1A is a micrograph showing the condition of a culture vessel after adhesion of a cell sheet to a cell sheet support.

FIG. 1B is a micrograph after Hoechst staining, showing the condition of a culture vessel after adhesion of a cell sheet to a cell sheet support.

FIG. 10 is a micrograph showing the condition of a culture vessel after adhesion of a cell sheet to CellShifter™.

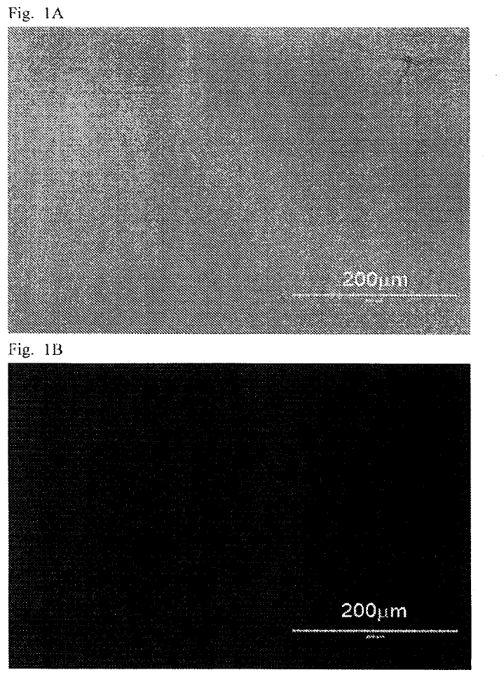

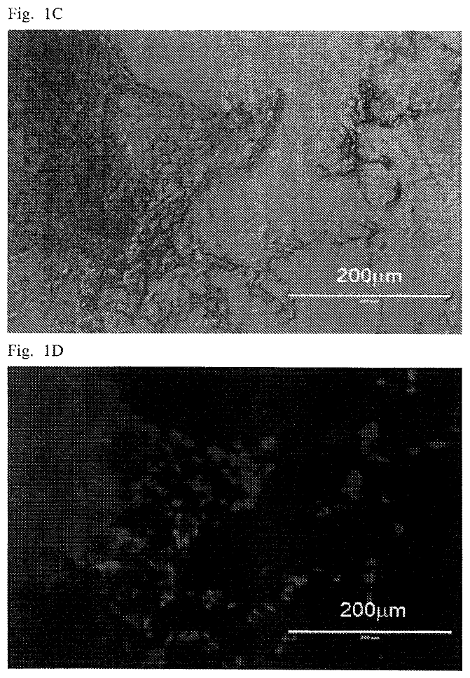

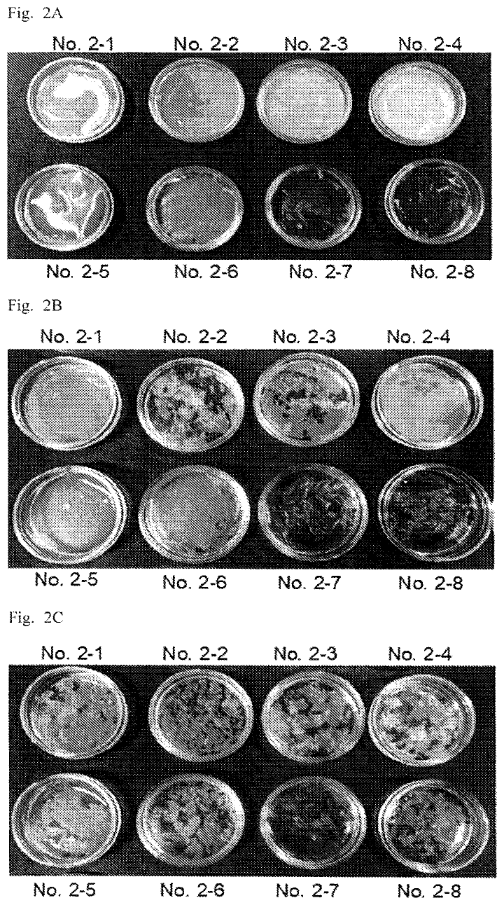

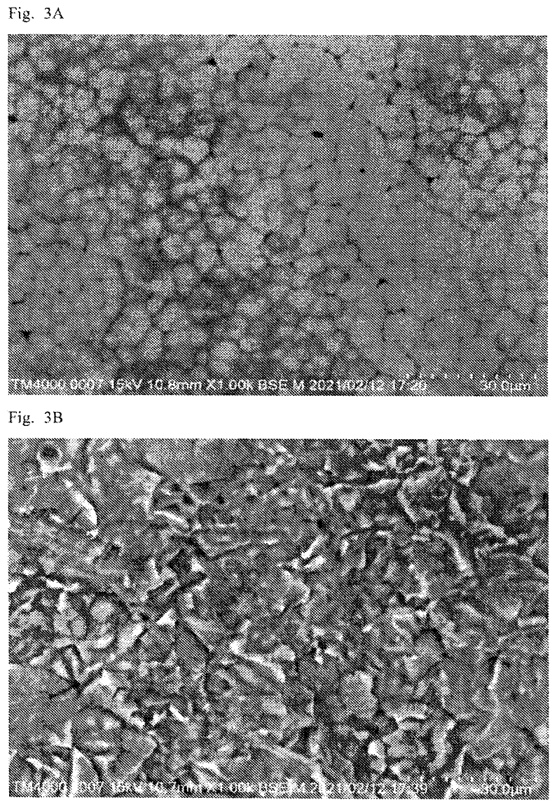

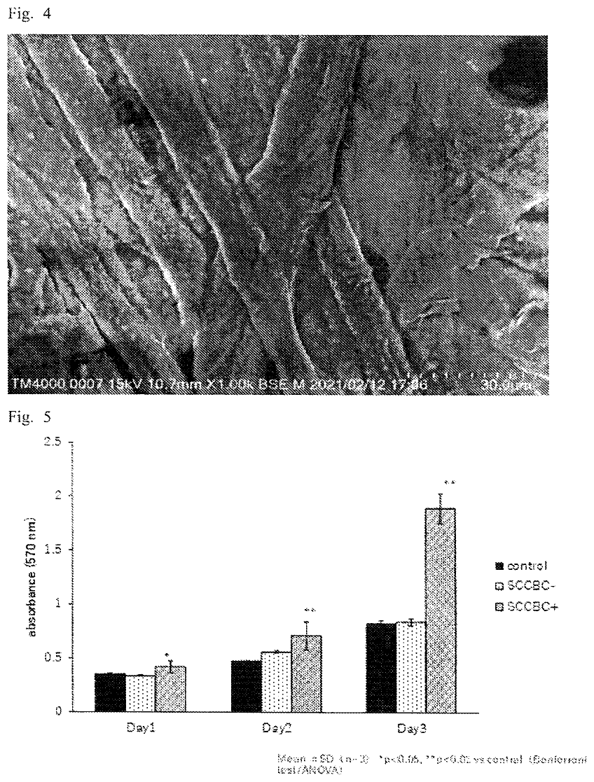

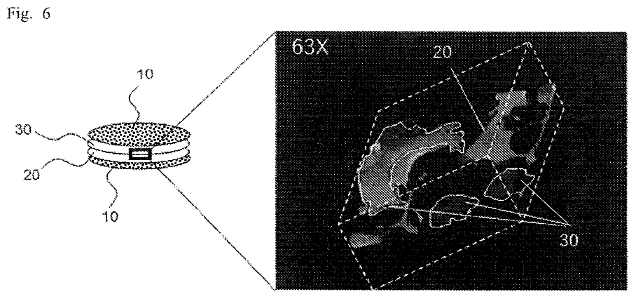

FIG. 1D is a micrograph after Hoechst staining, showing the condition of a culture vessel after adhesion of a cell sheet to CellShifter™.

FIG. 2A is an image showing hydrolyzability of a cell sheet support, which image was taken immediately after the start.

FIG. 2B is an image showing hydrolyzability of a cell sheet support, which image was taken at Week 2.

FIG. 2C is an image showing hydrolyzability of a cell sheet support, which image was taken at Week 4.

FIG. 3A is a magnified image of a surface of a cell sheet support.

FIG. 3B is a magnified image of a surface of a cell sheet support.

FIG. 4 is a magnified image of a surface of CellShifter™.

FIG. 5 is a diagram showing the growth rate of cultured cells on a cell sheet support.

FIG. 6 is a schematic diagram and a fluorescence micrograph showing the condition of the adhesion surface between different cell sheets layered on each other.

### MODE FOR CARRYING OUT THE INVENTION

In the present description, the term “step” includes not only an independent step, but also a step that is not clearly distinguishable from another step, as long as the desired purpose of the step can be achieved. Unless otherwise specified, when a plurality of substances corresponding to a certain component is present in a composition, the content of the component in the composition means the total amount of the plurality of substances present in the composition. Upper-limit values and lower-limit values of a numerical range described in the present description may be arbitrarily selected and combined. Embodiments of the present invention are described below in detail. The embodiments described below, however, are merely examples of cell sheet supports for realization of the technological thought of the present invention. Therefore, the present invention is not limited to the cell sheet supports described below.

Cell Sheet Support

The cell sheet support comprises a first polymer containing a structural unit derived from p-dioxanone, and is formed into a sheet shape. Since the cell sheet support contains the first polymer as a constituent component, the cell sheet support shows excellent adhesion to cultured cells and good biodegradability.

The first polymer, containing the structural unit derived from p-dioxanone, may be polydioxanone (poly(p-dioxanone); PDS), which is a homopolymer of p-dioxanone, or may be a copolymer of p-dioxanone and, for example, glycolic acid, lactic acid, or caproic acid. Preferably, the polydioxanone copolymer may be a copolymer of p-dioxanone and at least one kind of other monomers selected from the group consisting of glycolic acid, lactic acid, and caproic acid, or may be a copolymer of glycolic acid and p-dioxanone. The molar content ratio of the structural unit derived from p-dioxanone in the polydioxanone copolymer may be, for example, not less than 75%, preferably not less than 80%, or not less than 90%, with respect to the total number of moles of the structural unit. The polydioxanone copolymer may be a random copolymer or a block copolymer. The polydioxanone copolymer is preferably a block copolymer.

Polydioxanone (PDS) is a polymer prepared by the ring-opening polymerization of p-dioxanone. Compared to other biodegradable polymers, polydioxanone has superior absorbability, flexibility, and bendability in vivo, and low toxicity. Therefore, polydioxanone is used in a variety of biomedical applications such as sutures, meshes, staples, clips, implantable orthopedic devices, and drug delivery. Further, by using a copolymer of p-dioxanone and, for example, glycolic acid or lactic acid, not only the bendability derived from PDS, but also the biodegradability may be controlled.

The polydioxanone and the polydioxanone copolymer may be selected from commercially available products, when appropriate. More specifically, they are available from, for example, Sigma-Aldrich.

The cell sheet support may be composed only of the first polymer, or may also comprise a second polymer containing at least one selected from the group consisting of polylactic acid, polyglycolic acid, polycaproic acid, and copolymers thereof. By the inclusion of the second polymer, the flexibility, biodegradability, and the like of the cell sheet support may be easily controlled.

The second polymer may comprise at least one selected from the group consisting of polylactic acid, polyglycolic acid, and lactic acid/polyglycolic acid copolymers (PLGAs), or may comprise at least PLGA. The weight average molecular weight of the second polymer may be, for example, 1000 to 50,000, or may be preferably 5000 to 20,000. In cases where the second polymer is a copolymer, the biodegradation rate of the cell sheet support can be controlled by appropriately selecting the composition ratios of the monomers. For example, in cases where the second polymer is PLGA, the molar ratio of lactic acid to glycolic acid (L/G) may be, for example, 0.3 to 5, or may be preferably 1 to 4.

In cases where the cell sheet support comprises the first polymer and the second polymer, the content ratio of the first polymer to the total amount of the first polymer and the second polymer may be, for example, not less than 50% by mass, or may be preferably not less than 60% by mass or not less than 70% by mass. The upper limit of the content ratio of the first polymer to the total amount of the first polymer and the second polymer may be, for example, less than 100% by mass, or may be preferably not more than 95% by mass, not more than 90% by mass, or not more than 85% by mass.

The cell sheet support may be formed into a sheet shape. The planar shape of the cell sheet support may be appropriately selected in accordance with the purpose and the like, and may be, for example, any of a rectangular shape, a polygonal shape, a nearly circular shape, a nearly elliptical shape, and the like. The size of the cell sheet laminate may be appropriately selected in accordance with the purpose and the like. The size of the cell sheet laminate, in terms of the area, may be, for example, 1 cm² to 100 cm².

The average thickness of the cell sheet support may be appropriately selected from the viewpoint of biodegradability, ease of handling, and the like, or may be selected in accordance with the polymer composition. The average thickness of the cell sheet support may be, for example, not less than 10 μm and not more than 150 μm, preferably not less than 15 μm, not less than 20 μm, or not less than 30 μm. The upper limit of the average thickness may be, for example, not more than 140 μm, not more than 120 μm, or not more than 110 μm. The average thickness of the cell sheet support is calculated as the arithmetic average of the thicknesses at three arbitrary points.

The cell sheet support can be formed by molding a support-forming composition which is in the form of a solution containing a first polymer, a liquid medium, and, when necessary, a second polymer, into a membrane shape, and then removing the liquid medium. The liquid medium may be a solvent capable of dissolving the first polymer, and may be volatile. Specific examples of the liquid medium include hexafluoro-2-propanol (HFIP). The solid concentration of the support-forming composition may be, for example, 0.1% by mass to 5% by mass, or may be preferably 1% by mass to 4% by mass.

The cell sheet support can be formed into a desired shape by, for example, pouring the support-forming composition into a mold having the desired shape, and then removing the liquid medium. Further, the thickness can be controlled by adjusting the total solid content of the support-forming composition to be poured into the mold. Alternatively, the support-forming composition may be formed into an arbitrary shape by adding the composition dropwise onto a flat substrate without a mold. The materials of the mold and the substrate are not limited as long as the cell sheet support formed can be detached therefrom, and examples of the materials include glass, and resins such as polypropylene.

The method of removing the liquid medium may be selected in accordance with the properties of the liquid medium. More specifically, the liquid medium may be removed by evaporation at room temperature or under warming conditions. The cell sheet support may be formed in a porous shape, or may be formed into a non-porous, dense membrane shape.

Cell Sheet Laminate

The cell sheet laminate comprises: the cell sheet support; and a cell sheet placed on the cell sheet support. The cell sheet laminate may be produced by the production method described later.

Since the cell sheet support shows excellent adhesion to cultured cells, it is capable of allowing stable adhesion of a cell sheet at a high recovery rate, to retain the cell sheet on the cell sheet support. Therefore, the cell sheet support may be utilized to transfer a cell sheet formed on a cell culture vessel, from the culture vessel to a desired site. Even in cases where the site to which the cell sheet is to be transferred is a living body, removal of the cell sheet support after the transfer of the cell sheet is unnecessary because the cell sheet support is biodegradable. Further, on one side of the cell sheet support, a plurality of cell sheet supports each retaining a cell sheet may be layered to form a cell sheet laminate in which the plurality of cell sheets are layered through the cell sheet supports. By degradation of the cell sheet supports, the plurality of cell sheets become directly layered on each other to form a cell sheet laminate.

The cell sheet herein means a sheet-shaped aggregate of cultured cells obtained by culturing the cells on one side of a culture vessel. More specifically, the cell sheet may be a sheet-shaped aggregate of cultured cells obtained by culturing the cells to confluence on a cell culture dish.

The cells constituting the cell sheet are not limited in terms of the species and the tissue from which the cells are derived, as long as the cells are animal cells. Examples of the cells include cells immediately after collection from a living body, and established cell lines. Examples of the origin of the animal cells include mammals, including humans. The cells may be either somatic cells or stem cells.

In one aspect, the cell sheet laminate may also comprise a hydrophilic coating layer containing a hydrophilic polymer between the cell sheet support and the cell sheet.

Method of Producing Cell Sheet Laminate

The method of producing a cell sheet laminate comprises: a providing step of culturing cells on a temperature-responsive polymer layer, to provide a cell sheet; a layering step of layering the cell sheet support on the cell sheet; a temperature control step of detaching the cell sheet from the temperature-responsive polymer layer by a temperature change; and an adhesion step of allowing the cell sheet to adhere to the cell sheet support. Since the cell sheet is cultured on a temperature-responsive polymer layer, the cell sheet may be easily detached from the temperature-responsive polymer layer by giving a predetermined temperature change, and may be easily transferred to, and can adhere to, a cell sheet support having high affinity to cultured cells. By this, a cell sheet laminate in which the cell sheet is placed on the cell sheet support can be obtained.

In the providing step, cells are cultured on a temperature-responsive polymer to provide a cell sheet. More specifically, by culturing desired cells using a cell culture support having an area coated with a temperature-responsive polymer, a cell sheet is formed.

Examples of the material of the substrate to be coated with the temperature-responsive polymer constituting the cell culture support include polyvinyl resins such as polystyrene, polyethylene, and polypropylene; and glass. The shape, size, and the like of the substrate are not limited as long as the substrate enables the cell culture. They may be appropriately selected in accordance with the shape, size, and the like of the cell sheet of interest. The substrate may be appropriately selected from commercially available substrates for cell culture in accordance with the purpose and the like.

The temperature-responsive polymer is a polymer material whose adhesion to cultured cells changes reversibly depending on the ambient temperature. As the temperature-responsive polymer, a polymer whose adhesion to cultured cells decreases in an environment at a temperature lower than the cell culture temperature (hereinafter referred to as “first temperature-responsive polymer”) may be used. Example of the first temperature-responsive polymer include a polymer described in JP 2-211865 A. More specifically, a polymer obtained by homopolymerization or copolymerization of at least one kind of monomer selected from the group consisting of (meth)acrylamide compounds, alkyl-substituted (meth)acrylamide derivatives, and vinyl ether derivatives may be used. For example, poly(N-isopropylacrylamide) may be preferably used.

As the temperature-responsive polymer, a polymer whose adhesion to cultured cells decreases in an environment at a temperature slightly higher than the cell culture temperature (hereinafter referred to as “second temperature-responsive polymer”) may also be used. By the use of the second temperature-responsive polymer, low-temperature damage to the cultured cells in the detachment of the cell sheet can be effectively suppressed. Examples of the second temperature-responsive polymer include a polymer described in JP 2018-102296 A. More specifically, a copolymer comprising: a first constituent unit derived from (meth)acrylate containing an alkyl group having 14 to 22 carbon atoms; and a second constituent unit derived from (meth)acrylic acid; may be preferably used.

Specific examples of monomers forming the first constituent unit include (meth)acrylates containing a linear alkyl group, such as tetradecyl (meth)acrylate, hexadecyl (meth)acrylate, stearyl (meth)acrylate, eicosanyl (meth)acrylate, and behenyl (meth)acrylate.

The content molar ratio between the first constituent unit and the second constituent unit contained in the second temperature-responsive polymer (first constituent unit:second constituent unit) is, for example, 2:8 to 5:5, preferably 2:8 to 4:6. The second temperature-responsive polymer may be a random copolymer, or may be a block copolymer. The second temperature-responsive polymer is preferably a block copolymer.

The second temperature-responsive polymer may also contain a constituent unit other than the first constituent unit and the second constituent unit as long as the effect of the present invention does not deteriorate. The monomers that constitute the other constituent unit are not limited as long as the monomers are copolymerizable with a (meth)acrylate containing an alkyl group having 14 to 22 carbon atoms, and with (meth)acrylic acid. Examples of such monomers include styrene, and (meth)acrylates containing an alkyl group having not more than 12 carbon atoms, such as butyl acrylate.

The molecular weight of the second temperature-responsive polymer may be 3,500 to 200,000, preferably 6,000 to 70,000, in terms of the weight average molecular weight (Mw). The polydispersity (Mw/Mn) may be, for example, 1.05 to 15, preferably 1.2 to 2. The weight average molecular weight and the polydispersity may be determined in terms of polystyrene by using GPC.

In cases where the second temperature-responsive polymer is a block copolymer, the weight average molecular weight of the first constituent unit part may be, for example, not less than 500, not less than 1,000, not less than 2,000, not less than 3,000, not less than 4,000, or not less than 5,000, and may be, for example, not more than 100,000, not more than 50,000, not more than 10,000, not more than 8,000, or not more than 7,000. The weight average molecular weight of the second constituent unit part may be, for example, not less than 500, not less than 1,000, or not less than 2,000, and may be, for example, not more than 100,000, not more than 20,000, or not more than 15,000. The degree of polymerization of the first constituent unit part may be, for example, not less than 2, not less than 5, or not less than 10, and may be, for example, not more than 800, not more than 500, not more than 100, or not more than 50. The degree of polymerization of the second constituent unit part may be, for example, not less than 5, not less than 10, or not less than 30, and may be, for example, not more than 800, not more than 500, or not more than 100.

The amount of temperature-responsive polymer placed on the surface of the substrate may be, for example, 30 μg/cm² to 160 μg/cm², preferably 50 μg/cm² to 80 μg/cm². A single kind of polymer may be used alone, or two or more kinds of polymers having different constitutions may be used in combination.

The cells cultured on the cell culture support are not limited in terms of the species and the tissue from which the cells are derived, as long as the cells are animal cells. Examples of the cells include cells immediately after collection from a living body, and established cell lines. Examples of the origin of the animal cells include mammals, including humans. The cells may be either somatic cells or stem cells.

For the culture of the cells, a medium that is usually employed may be used. The medium used in the culture is not limited as long as the medium is commonly employed for a culture of animal cells. Examples of the medium include serum-free basal culture media (standard culture media), such as RPMI medium, Dulbecco's Modified Eagle's Medium (DMEM), MEM medium, and F12 medium. This medium may be supplemented with serum for promoting the cell growth, or with, for example, a cell growth factor such as FGF, EGF, or PDGF, or a known serum component such as transferrin, as an alternative to the serum. In cases where the medium is supplemented with serum, the concentration of the serum may be changed in accordance with the culture condition at that time. The concentration may be, for example, 5% by volume to 10% by volume. The medium may be supplemented also with various vitamins, antibiotics such as streptomycin, differentiation inducers, and/or the like.

The density of the cells seeded on the cell culture support is not limited as long as the cell sheet can be formed. The density may be appropriately selected in accordance with the cell type and the like. The density per culture area may be, for example, 3×10⁴ to 6×10⁴ cells/cm².

The cell culture conditions may be appropriately selected depending on the cells. The culture temperature may be, for example, 35° C. to 37° C. The cell culture may be carried out in an incubator at a CO₂ concentration of 5%. In the formation of cell sheets derived from various cells, the culture may be carried out to confluence under normal culture conditions for about 3 to 5 days. The formation of the cell sheets can be confirmed by observation under the microscope.

In the layering step, the cell sheet support described above is layered on the provided cell sheet. The size of the cell sheet support to be layered on the cell sheet may be almost the same as the size of the provided cell sheet, or may be larger than the size of the cell sheet. In cases where a cell sheet support larger than the cell sheet is used, a cell sheet support having an area about 1% to 10% larger than the area of the cell sheet may be used.

In the layering step, at least part of the medium covering the cell sheet may be removed before the layering of the cell sheet support on the cell sheet. By removing the medium to expose the cell sheet, the cell sheet can be easily brought into contact with the cell sheet support. In the layering step, the cell sheet support may be layered on the cell sheet, and then a retention time may be provided for maintaining the state where the cell sheet is in contact with the cell sheet support. The retention time may be, for example, 5 minutes to 30 minutes.

Temperature Control Step

In the temperature control step, the cell sheet is detached from the temperature-responsive polymer by a temperature change. In cases where the temperature-responsive polymer is a first temperature-responsive polymer, the temperature change is changing the temperature from the culture temperature of the cell sheet to a temperature lower than the culture temperature. More specifically, by changing the temperature to, for example, 20° C. to 25° C., the cell sheet can be detached from the temperature-responsive polymer layer. In cases where the temperature-responsive polymer is a second temperature-responsive polymer, the temperature change is changing the temperature from the culture temperature of the cell sheet to a temperature higher than the culture temperature. More specifically, by changing the temperature to, for example, 38° C. to 45° C., the cell sheet can be detached from the temperature-responsive polymer layer. In the temperature control step, the time during which the changed temperature is maintained may be, for example, 30 minutes to 60 minutes.

The temperature control step may be carried out after the layering step, or may be carried out before the layering step. In cases where the temperature is controlled after the layering step, the cell sheet that has adhered to the cell sheet support during the layering step is detached from the temperature-responsive polymer, to obtain a cell sheet laminate in the free state. In cases where the temperature is controlled before the layering step, the cell sheet support is layered on the cell sheet detached from the temperature-responsive polymer, to obtain a cell sheet laminate in the free state.

Adhesion Step

In the adhesion step, the cell sheet is allowed to adhere to the cell sheet support, to obtain a cell sheet laminate. The cell sheet support has high affinity to cultured cells. Therefore, when the cell sheet support is layered on, and brought into contact with, the cell sheet, the cell sheet adheres to the cell sheet support. The adhesion step may be carried out as a layering step before the temperature control step, or may be carried out as a layering step after the temperature control step.

Another aspect of the method of producing a cell sheet laminate is a method of producing a cell sheet laminate, the method comprising forming a cell sheet by culturing cells on the cell sheet support described above having a surface on which a hydrophilic coating layer containing a hydrophilic polymer is formed. Another aspect of the method of producing a cell sheet laminate is a method of producing a cell sheet laminate, the method comprising: forming a hydrophilic coating layer containing a hydrophilic polymer on the cell sheet support described above; and culturing cells on the hydrophilic coating layer, to form a cell sheet.

Examples of the hydrophilic polymer contained in the hydrophilic coating layer include synthetic polymers such as the temperature-responsive polymer described above; and tissue-derived polymers such as type I collagen, type III collagen, type IV collagen, type V collagen, laminin, polylysine, and extracellular matrix (ECM) hydrogel. The hydrophilic coating layer may contain one kind of hydrophilic polymer alone, or two or more kinds of hydrophilic polymers in combination. The hydrophilic polymer preferably comprises at least one selected from the group consisting of, for example, a copolymer containing a constituent unit derived from (meth)acrylate containing an alkyl group having 14 to 22 carbon atoms, and a constituent unit derived from (meth)acrylic acid; type I collagen; type III collagen; type IV collagen; type V collagen; laminin; polylysine; and ECM hydrogel; or may comprise at least a copolymer containing a constituent unit derived from (meth)acrylate containing an alkyl group having 14 to 22 carbon atoms, and a constituent unit derived from (meth)acrylic acid.

The thickness of the hydrophilic coating layer may be, for example, 10 nm to 2000 μm, or 20 nm to 1000 μm. In cases where the hydrophilic polymer contained in the hydrophilic coating layer is a synthetic polymer such as the above-described temperature-responsive polymer; or a tissue-derived polymer such as collagen, laminin, or polylysine; the thickness of the hydrophilic coating layer may be preferably not less than 20 nm or not less than 50 nm, and preferably not more than 500 nm or not more than 200 nm. In cases where the hydrophilic polymer is a hydrogel having an ECM composition, the thickness of the hydrophilic coating layer may be preferably not less than 100 μm or not less than 300 μm, and preferably not more than 1000 μm or not more than 500 μm. The hydrophilic coating layer may be placed only on one side of the cell sheet support, or may be placed on both sides.

The hydrophilic coating layer can be formed on the cell sheet support by, for example, applying a solution of the hydrophilic polymer to the surface(s) of the cell sheet support, and removing at least part of the solvent.

The method of culturing cells on the cell sheet support described above on which the hydrophilic coating layer is formed may be carried out by application of the same method as the method of forming the cell sheet by culturing cells on the temperature-responsive polymer described above.

Method of Transferring Cell Sheet

The method of transferring a cell sheet comprises: a providing step of culturing cells on a temperature-responsive polymer layer, to provide a cell sheet; a layering step of layering the cell sheet support described above on the cell sheet; a temperature control step of detaching the cell sheet from the temperature-responsive polymer layer by a temperature change; an adhesion step of allowing the cell sheet to adhere to the cell sheet support; and a transfer step of bringing the cell sheet on the cell sheet support into contact with a target site.

By transferring the cell sheet together with the cell sheet support to the target site to bring the cell sheet into contact with the target site, damage to the cell sheet can be suppressed, and ease of handling of the cell sheet can be improved.

The providing step, layering step, temperature control step, and adhesion step in the method of transferring a cell sheet have the same meanings as those in the above-described method of producing a cell sheet laminate.

In the transfer step, the cell sheet on the cell sheet support is transferred to the target site, and brought into contact with the target site. In other words, the cell sheet laminate obtained by the adhesion step is transferred to the target site, and the cell sheet on the cell sheet support is brought into contact with the target site. The target site may be either in vivo or in vivo. Examples of the in vivo target site include another cell sheet, a laminate of a plurality of cell sheets, another cell sheet laminate, and a tissue or an organ collected from a living body. The target site may also be a tissue or an organ in vitro.

Another aspect of the method of transferring a cell sheet may comprise: forming a cell sheet by culturing cells on the cell sheet support described above having a surface on which a hydrophilic coating layer containing a hydrophilic polymer is formed; and bringing the cell sheet on the cell sheet support into contact with a target site.

### EXAMPLES

The present invention is described below more concretely by way of Examples. However, the present invention is not limited to these Examples.

### Example 1

Polydioxanone (PDS; manufactured by Sigma-Aldrich) as a first polymer, and a lactic acid/glycolic acid copolymer (PLGA5005; manufactured by FUJIFILM Wako Pure Chemical Co., Ltd.; L/G 50%, Mw 5000) as a second polymer were provided. The PDS and the PLGA5005 were mixed as shown in Table 1, and 1 mL of hexafluoro-2-propanol (manufactured by Sigma-Aldrich) was added to the resulting mixture, followed by mixing the mixture with shaking to prepare a polymer solution. The polymer solution was spread on a glass plate using a pipette, and then dried by being left to stand at room temperature for about 1 hour, followed by detachment of the resulting product from the glass plate with tweezers, to obtain cell sheet supports Nos. 1-1 to 1-3.

Average Thickness and Detachability

Each cell sheet support obtained in Example 1 was subjected to measurement of the thickness at three points using a digital caliper (DC-10, TOPMIGHTY), and the average thickness was calculated as the arithmetic average of the measured values. In addition, detachability from the glass plate was visually evaluated. The results are shown in Table 1.

TABLE 1

| | | | PDS | Average | |
|---|---|---|---|---|---|
| | PDS | PLGA5005 | raito | thickness | Detach- |
| No. | (mg) | (mg) | (%) | (mm) | ability |
| 1-1 | 40 | — | 100 | 40 | Good |
| 1-2 | 30 | 10 | 75 | 40 | Good |
| 1-3 | 20 | 20 | 50 | 30 | Pass |

Cell Recovery Rate

Adhesion of each cell sheet support obtained in Example 1 to a cell sheet was evaluated as follows. The results are shown in FIG. 1.

By referring to the description in JP 2018-102296 A, a temperature-responsive polymer composed of tetradecylacrylate (TDA) and acrylic acid (AA), having a constitution of a weight average molecular weight of TDA:AA=10,000:10,000 was synthesized. The synthesized temperature-responsive polymer was dissolved to 0.05% (w/v) in dimethyl sulfoxide (DMSO), to prepare a composition for coating a cell culture dish.

In a 35-mm untreated polystyrene (PS) dish (IWAKI #1000-035), 1.5 mL of the obtained composition was added dropwise, and then the dish was left to stand for 2 hours at room temperature, followed by rinsing the dish by addition of 2 mL of PBS, to prepare a culture vessel that is a cell culture support having a surface to which the temperature-responsive polymer adhered. Subsequently, Myocyte Basal Medium (supplemented with 5% FCS, hEGF (0.5 ng/mL), hbFGF (2 ng/mL), and Insulin (5 μg/mL)) was added to the prepared cell culture support, and a human cardiomyocyte cell line (HCM-c) was plated at 1×10 5 cells/dish. Cell culture was carried out for 28 days in an incubator at 37° C. at a CO₂ concentration of 5%, to form a cell sheet. Subsequently, the culture was shaken at 40° C. for 30 to 60 minutes, and then the medium was removed by aspiration, followed by layering each cell sheet support obtained in Example 1 on the cell sheet. After leaving the layered cell sheet support to stand for 5 minutes, the cell sheet support, to which the cell sheet adhered, was removed from the cell culture support, and the cell sheet was collected. After diluting 1 μL of Hoechst 33342 reagent with 500 μL of PBS, the resulting dilution was added to each culture dish after the collection of the cell sheet. The culture dish was then left to stand for 30 minutes in an incubator at 37° C. at a CO₂ concentration of 5%. The cells that remained in the culture vessel after the collection were visualized by fluorescence by Hoechst staining, and observation was carried out under the microscope.

FIG. 1(A) is a micrograph showing the condition of the culture vessel after the collection of the cell sheet using the cell sheet support No. 1-2, and FIG. 1(B) is a micrograph after Hoechst staining (405 nm). FIG. 1(C) is a micrograph showing the condition of the culture vessel after the collection of the cell sheet using CellShifter™ (manufactured by CellSeed Inc.), and FIG. 1(D) is a micrograph after Hoechst staining (405 nm). It can be seen that, by using the cell sheet support, the cell sheet can be collected with a good recovery rate without leaving cultured cells in the culture vessel.

### Example 2

Cell sheet supports Nos. 2-1 to 2-8 were obtained in the same manner as in Example 1 except that PLGA5010 (L/G 50%, Mw 10,000), PLGA5020 (L/G 50%, Mw 20,000), PLGA0010 (L/G 100%, Mw 10,000), and PLGA7510 (L/G 75%, Mw 10,000) (all of which were manufactured by FUJIFILM Wako Pure Chemical Co., Ltd.) were provided as second polymers in addition to PLGA5005, and that the compositions shown in Table 2 were employed.

TABLE 2

| | PDS | | PDS raito |
|---|---|---|---|
| No. | (mg) | PLGA | (%) |
| 2-1 | 40 | — | 100 |
| 2-2 | 30 | PLGA5005 | 75 |
| | | 10 mg | |
| 2-3 | 30 | PLGA5010 | 75 |
| | | 10 mg | |
| 2-4 | 30 | PLGA5020 | 75 |
| | | 10 mg | |
| 2-5 | 40 | — | 100 |
| 2-6 | 30 | PLGA0010 | 75 |
| | | 10 mg | |
| 2-7 | 30 | PLGA7510 | 75 |
| | | 10 mg | |
| 2-8 | 30 | PLGA5010 | 75 |
| | | 10 mg | |

Hydrolyzability

Each cell sheet support was cut into the shape of the bottom surface of a 35-mm dish, and placed on the bottom surface of the dish. After adding 1.5 mL of PBS dropwise thereto, the dish was left to stand for 4 weeks under conditions at 37° C. while observation was carried out over time. The results are shown in FIG. 2.

FIG. 2 (A) shows an image obtained immediately after the dropwise addition (Day 0). FIG. 2 (B) shows an image obtained at Week 2 (Day 14). FIG. 2(C) shows an image obtained at Week 4 (Day 28). It was found that, by adjusting the ratio between the lactic acid and the glycolic acid (L/G), and the molecular weight, of the PLGA contained in the cell sheet support, the time required for the degradation and the flexibility can be controlled. More specifically, it was found that, as the molecular weight increases, the time required for the degradation tends to increase, and that, as the molecular weight increases, the cell sheet support tends to be harder.

### Example 3

A recessed portion having a diameter of 31 mm was formed on a glass plate, to provide a mold for preparing a cell sheet support. Cell sheet supports Nos. 3-1 to 3-6 were obtained in the same manner as in Example 1 except that the compositions shown in Table 3 were employed and that the polymer solution was spread in the mold.

TABLE 3

| | | | PDS | Average |
|---|---|---|---|---|
| | PDS | PLGA5010 | ratio | thickness |
| No. | (mg) | (mg) | (%) | (mm) |
| 3-1 | 40 | — | 100 | 105 |
| 3-2 | 20 | — | 100 | 40 |
| 3-3 | 10 | — | 100 | 13 |
| 3-4 | 30 | 10 | 75 | 107 |
| 3-5 | 15 | 5 | 75 | 47 |
| 3-6 | 7.5 | 2.5 | 75 | 23 |

Cell Recovery Rate

By referring to the description in JP 2018-102296 A, a temperature-responsive polymer composed of tetradecylacrylate (TDA) and acrylic acid (AA), having a constitution of a weight average molecular weight of TDA:AA=10,000:10,000 was synthesized. The obtained temperature-responsive polymer was dissolved to 0.05% (w/v) in dimethyl sulfoxide (DMSO) to prepare a composition for coating a cell culture dish. In a 35-mm untreated polystyrene (PS) dish (IWAKI #1000-035), 1.5 mL of the obtained composition was added dropwise, and then the dish was left to stand for 2 hours at room temperature, followed by rinsing the dish by addition of 2 mL of PBS, to prepare a cell culture support having a surface to which the temperature-responsive polymer adhered.

Onto the cell culture support, DMEM medium (supplemented with 10% FCS) was added as a medium, and the human oral cancer cell line NA was plated thereon at 3×10 5 cells/dish, followed by culturing the cells to confluence for 7 days under conditions at 37° C. and 5% CO₂, to form a cell sheet on the temperature-responsive polymer layer.

The cell culture support, together with the cell sheet formed thereon, was warmed at 40° C. for 30 minutes to detach the cell sheet from the temperature-responsive polymer layer. Subsequently, the medium was removed, and the cell sheet support was layered on the cell sheet, followed by leaving the layered cell sheet support to stand for 5 minutes. Thereafter, the cell sheet support, to which the cell sheet adhered, was removed from the cell culture support. The cells that remained in the cell culture support were subjected to Hoechst staining, and the cell residual ratio was calculated based on the fluorescence intensity, using the fluorescence intensity before the detachment of the cell sheet as the reference (100%). The results are shown in Table 4. The Reference Example used CellShifter™.

Flexibility

Flexibility of the cell sheet support was evaluated according to the following scoring criteria based on calculation of the ratio of the area of the cell sheet support adhering to the bottom surface of the cell culture support to the bottom area of the cell culture support as observed when the cell sheet support was layered on the cell sheet. The results are shown in Table 4.

Scoring Criteria
A: Not less than 90% of the bottom area
B: Not less than 60% and less than 90% of the bottom area
C: Not less than 50% and less than 60% of the bottom area
D: Not less than 30% and less than 50% of the bottom area
E: Less than 30% of the bottom area
F: No adhesion

TABLE 4

| | Cell recovery | |
|---|---|---|
| No. | rate (%) | Flexibility |
| 3-1 | 79.5 | C |
| 3-2 | 78.9 | B |
| 3-3 | 78.5 | A |
| 3-4 | 77.7 | C |
| 3-5 | 78.5 | B |
| 3-6 | 78.4 | A |
| Comparaitve | 7.3 | — |
| Example | | |

Surface Observation

For each of the cell sheet supports No. 3-2 and No. 3-5 obtained in Example 3, the surface of the cell sheet support was observed using a tabletop microscope (Miniscope® TM4000 Plus; manufactured by Hitachi, Ltd.), to acquire a magnified image (×1000). The results are shown in FIG. 3. FIG. 4 is a magnified image of the surface of CellShifter™ in Reference Example.

FIG. 3(A) is a magnified image of No. 3-2, and FIG. 3 (B) is a magnified image of No. 3-5, wherein each image was taken near the center of the surface. As shown in FIG. 3 and FIG. 4, it can be seen that, while Cellshifter™ is fibrous, the PDS film and the PDS/PLGA film, which are cell sheet supports, have solid structures that are neither fibrous nor porous. It can also be seen that the PDS film has a structure in which polymers are arranged in a cobblestone pattern, and that the PDS/PLGA film has a ruffled structure.

Moisture Absorption

The rate of moisture absorption of the cell sheet support was calculated by the following method. The dry weight A (g) was measured after drying the cell sheet support in a drying apparatus (SPH-10N, manufactured by IKEDARIKA) for not less than 3 hours. The dried cell sheet support was left to stand for 1 hour in an environment at 40° C. at 90% RH (SCA-30D, manufactured by ASTEC Co., Ltd.), and then the weight B (g) after moisture absorption was measured. The rate of moisture absorption (%) was calculated by the following equation. The results are shown in Table 5.

Rate of moisture absorption (%)=(B−A)/A×100

TABLE 5

| | Rate of moisture |
|---|---|
| No. | absorption (%) |
| 3-1 | 1.64 |
| 3-2 | 1.69 |
| 3-3 | 1.74 |
| 3-4 | 2.14 |
| 3-5 | 2.10 |
| 3-6 | 1.45 |
| Comparaitve | 7.71 |
| Example | |

The contact angles of the cell sheet supports of No. 3-2 and No. 3-5 were measured by the following method. Using a contact angle meter (DropMaster DM-301, manufactured by Kyowa Interface Science Co., Ltd.), the contact angle was measured by the dropping method, in which 1 μL of purified water was dropped onto each cell sheet support. The results are shown in Table 6.

TABLE 6

| No. | Contact angle θ° |
|---|---|
| 3-2 | 66.1 ± 1.8 |
| 3-5 | 60.2 ± 3.3 |
| Comparative | 23.4 ± 0.9 |
| Example | |

Average ± SD (n = 5)

Compared to CellShifter™, the cell sheet supports had lower rates of moisture absorption. Further, compared to CellShifter™, the cell sheet supports had larger contact angles, indicating that the cell sheet supports have contact angles suitable for cell culture. It was thus suggested that the large contact angles (high surface tensions) of the cell sheet supports enhanced the adsorption efficiency of the cell sheet.

### Example 4

To 40 mg of a dioxanone/glycolic acid copolymer (poly(dioxanone-co-glycolide), (90:10); manufactured by Sigma-Aldrich), 1 mL of hexafluoro-2-propanol (manufactured by Sigma-Aldrich) was added, and the copolymer was dissolved to prepare a polymer solution. The polymer solution was spread on a glass plate using a pipette, and then dried by being left to stand at room temperature for about 1 hour, followed by detachment of the resulting product from the glass plate with tweezers, to obtain a cell sheet support of Example 4.

For the obtained cell sheet support of Example 4, the cell recovery rate, the flexibility, and the average thickness were evaluated in the same manner as described above. As a result, the cell recovery rate was 78.5%; the flexibility score was B; and the average thickness was 30 μm.

### Example 5

The cell sheet support obtained in No. 1-2 of Example 1 was cut into the same size as the bottom surface of each well of an uncoated 96-well plate, and placed on the bottom surface of each well. A 0.05% (w/v) solution of the temperature-responsive polymer obtained as described above (hereinafter referred to as SCCBC) was added to the well, and the plate was left to stand at room temperature for 2 hours, followed by washing with PBS solution. The cell sheet support to which the temperature-responsive polymer adhered was removed, and transferred to a new well. Subsequently, the mouse melanoma cell line B16/BL6GFP was plated on the cell sheet support at 1×10⁴ cells/well, and culture was performed for 72 hours. A WST-8 assay (cell counting kit-8 (CCK-8): Dojindo Laboratories) was performed 24 hours, 48 hours, and 72 hours after the start of the culture, to evaluate the cell growth rate. The results are shown as SCCBC+ in FIG. 5. A result obtained by performing cell culture without placing the cell sheet support was used as a control.

### COMPARATIVE EXAMPLE

Cell culture was carried out in the same manner as described above except that the cell sheet support having no temperature-responsive polymer adhering thereto was placed on the well. The results are shown as SCCBC− in FIG. 5.

It can be seen from FIG. 5 that, by culturing the cells on the cell sheet support having a surface to which the temperature-responsive polymer as a hydrophilic polymer is adhering, the cells can be allowed to grow favorably.

### Example 6

Onto a cell culture support having a surface to which the temperature-responsive polymer is adhering, prepared in the same manner as described above, DMEM medium (supplemented with 10% FCS) was added as a medium, and the mouse melanoma cell line B16/BL6 GFP (green fluorescence) was plated thereon at 3×10⁵ cells/dish, followed by culturing the cells to confluence for 5 days under conditions at 37° C. and 5% CO₂, to form a cell sheet on the temperature-responsive polymer layer. The cell culture support, together with the cell sheet formed thereon, was warmed at 40° C. for 30 minutes to detach the cell sheet from the temperature-responsive polymer layer. Subsequently, the medium was removed, and a cell sheet support was layered on the cell sheet, followed by leaving the layered cell sheet support to stand for 5 minutes. Thereafter, a cell sheet laminate in which the cell sheet of the mouse melanoma cell line is adhering to the cell sheet support was obtained to provide cell sheet laminate A.

A cell sheet laminate in which a cell sheet of a human cervical cancer cell line is adhering to the cell sheet support was obtained in the same manner as described above except that the human cervical cancer cell line HeLa tdTomato (red fluorescence) was used instead of the mouse melanoma cell line, to provide cell sheet laminate B.

The cell sheet support A and the cell sheet support B were layered such that their cell-sheet sides faced each other. Continuous culture was carried out for 3 days to obtain a laminate of the cell sheets in which the different cell sheets are layered. The obtained laminate of the different cell sheets was observed at the adhesion surface portion between the two kinds of cell sheets, using a confocal laser fluorescence microscope (LSM710, manufactured by ZEISS). The result is shown in FIG. 6 together with a schematic diagram of the laminate.

As shown in the schematic diagram in FIG. 6, the laminate comprises a cell sheet support 10, a cell sheet 20 of the mouse melanoma cell line, a cell sheet 30 of the human cervical cancer cell line, and a cell sheet support 10, which are arranged in this order. It can also be seen from FIG. 6 that the two different kinds of cell sheets show mixing of the different cells at the contact surface between the sheets, indicating that adhesion was achieved to form a laminate sheet.

The disclosure of Japanese patent application No. 2021-023164 (filing date: Feb. 17, 2021) is incorporated herein by reference in its entirety. All documents, patent applications, and technical standards cited in the present description are incorporated herein by reference to the same extent as in cases where the individual documents, patent applications, and technical standards are specifically and individually described to be incorporated by reference.

1. A cell sheet support comprising a first polymer containing a structural unit derived from p-dioxanone.

2. The cell sheet support according to claim 1, further comprising a second polymer containing at least one selected from the group consisting of polylactic acid, polyglycolic acid, polycaproic acid, and copolymers thereof.

3. The cell sheet support according to claim 2, wherein the content ratio of the first polymer to the total amount of the first polymer and the second polymer is not less than 50% by mass.

4. The cell sheet support according to claim 1, which has a sheet shape having an average thickness of not less than 10 μm and not more than 150 μm.

5. The cell sheet support according to claim 1, for use in transfer of a cell sheet.

6. The cell sheet support according to claim 1, for use in layering of a cell sheet.

7. A cell sheet laminate comprising:
the cell sheet support according to claim 1; and
a cell sheet placed on the cell sheet support.

8. A method of producing a cell sheet laminate, the method comprising:
culturing cells on a temperature-responsive polymer layer, to provide a cell sheet;
layering the cell sheet support according to claim 1 on the cell sheet;
detaching the cell sheet from the temperature-responsive polymer layer by a temperature change; and
allowing the cell sheet to adhere to the cell sheet support.

9. A method of transferring a cell sheet, the method comprising:
culturing cells on a temperature-responsive polymer layer, to provide a cell sheet;
layering the cell sheet support according to claim 1 on the cell sheet;
detaching the cell sheet from the temperature-responsive polymer layer by a temperature change;
allowing the cell sheet to adhere to the cell sheet support; and
bringing the cell sheet on the cell sheet support into contact with a target site.

10. The method according to claim 8, wherein the temperature-responsive polymer is a copolymer comprising a constituent unit derived from (meth)acrylate containing an alkyl group having 14 to 22 carbon atoms, and a constituent unit derived from (meth)acrylic acid.

11. The method according to claim 8, wherein the temperature change includes maintaining a temperature higher than the temperature at which the cells are cultured.

12. A method of producing a cell sheet laminate, the method comprising forming a cell sheet by culturing cells on a cell sheet support according to claim 1 having a surface on which a hydrophilic coating layer containing a hydrophilic polymer is formed.

13. A method of transferring a cell sheet, the method comprising:
forming a cell sheet by culturing cells on a cell sheet support according to claim 1 having a surface on which a hydrophilic coating layer containing a hydrophilic polymer is formed; and
bringing the cell sheet on the cell sheet support into contact with a target site.

14. The method according to claim 12, wherein the hydrophilic polymer comprises at least one selected from the group consisting of, for example, a copolymer containing a constituent unit derived from (meth)acrylate containing an alkyl group having 14 to 22 carbon atoms, and a constituent unit derived from (meth)acrylic acid; type I collagen; type III collagen; type IV collagen; type V collagen; laminin; polylysine; and ECM hydrogel.

15. The method according to claim 13, wherein the hydrophilic polymer comprises at least one selected from the group consisting of, for example, a copolymer containing a constituent unit derived from (meth)acrylate containing an alkyl group having 14 to 22 carbon atoms, and a constituent unit derived from (meth)acrylic acid; type I collagen; type III collagen; type IV collagen; type V collagen; laminin; polylysine; and ECM hydrogel.

16. The method according to claim 9, wherein the temperature-responsive polymer is a copolymer comprising a constituent unit derived from (meth)acrylate containing an alkyl group having 14 to 22 carbon atoms, and a constituent unit derived from (meth)acrylic acid.
